(19) United States
(12) Patent Application Publication
(10) Pub. No.: US 2024/0398832 A1
(43) Pub. Date: Dec. 5, 2024

(54) CDK4 AND 6 INHIBITOR IN COMBINATION WITH FULVESTRANT FOR THE TREATMENT OF HORMONE RECEPTOR-POSITIVE, HUMAN EPIDERMAL GROWTH FACTOR RECEPTOR 2-NEGATIVE ADVANCED OR METASTATIC BREAST CANCER IN PATIENTS PREVIOUSLY TREATED WITH A CDK4 AND 6 INHIBITOR

(71) Applicant: Eli Lilly and Company, Indianapolis, IN (US)

(72) Inventors: Lacey Morgan LITCHFIELD, Indianapolis, IN (US); Claudia MORATO GUIMARAES, Carmel, IN (US)

(21) Appl. No.: 18/717,475

(22) PCT Filed: Dec. 7, 2022
(86) PCT No.: PCT/US2022/052071

§ 371 Date: Jun. 7, 2024

Related U.S. Application Data
(60) Provisional application No. 63/321,218, filed on Mar. 18, 2022.
(60) Provisional application No. 63/288,179, filed on Dec. 10, 2021.

(51) Int. Cl. A61K 31/565; A61K 31/506; A61K 31/519; A61P 35/00

(52) U.S. Cl. CPC A61K 31/565; A61K 31/506; A61K 31/519; A61P 35/00

(57) ABSTRACT

Disclosed are methods, uses, and combinations for treating hormone receptor-positive (HR+), human epidermal growth factor receptor 2-negative (HIER2−) advanced or metastatic breast cancer in patients previously treated with a CDK4 and 6 inhibitor, the methods, uses, and combinations including the administration of a CDK4 and 6 inhibitor in combination with fulvestrant. The methods uses and combinations may include a CDK4 and 6 inhibitor such as palbociclib, ribociclib, or abemaciclib with fulvestrant.

### FIELD

The disclosure relates to the field of treatment of hormone receptor-positive (HR+), human epidermal growth factor receptor 2-negative (HER2−) advanced or metastatic breast cancer in patients previously treated with a CDK4 and 6 inhibitor.

### BACKGROUND

The combination therapy of CDK4 and 6 inhibitors with an endocrine therapy (ET) as a first-line treatment of locally advanced or metastatic hormone receptor positive (HR+), human epidermal growth factor receptor 2 negative (HER2−) breast cancer has dramatically improved outcomes (Finn et al. 2016; Tripathy et al. 2018; Johnston et al. 2019). Nevertheless, these therapies are not curative, and most metastatic breast cancer patients will experience disease progression. More recently, abemaciclib has shown significant improvement in invasive disease-free survival (IDFS) and distant relapse-free survival (DRFS) in the adjuvant setting (Johnston et al. 2020) in patients with early breast cancer at high risk of recurrence. As the use of CDK4 and 6 inhibitors increases in earlier lines of therapy, there exists a need for additional methods for treating patients who experience disease progression or recurrence on or after the CDK4 and 6-based therapy.

### SUMMARY

In an aspect, the disclosure provides a method of treating a patient with hormone receptor-positive (HR+), human epidermal growth factor receptor 2-negative (HER2−) advanced or metastatic breast cancer who has been previously treated with a CDK4 and 6 inhibitor-containing therapy, the method comprising administering to the patient a CDK4 and 6 inhibitor in combination with fulvestrant. In some further embodiments of this aspect, the method comprises administering to the patient a CDK4 and 6 inhibitor comprising abemaciclib, palbociclib, or ribociclib in combination with fulvestrant. In some preferred embodiments, the method comprises administering to the patient abemaciclib in combination with fulvestrant. In alternative embodiments, the method comprises administering to the patient palbociclib in combination with fulvestrant. In another embodiments, the method comprises administering to the patient ribociclib in combination with fulvestrant.

In some embodiments of this aspect, the patient may have been administered a prior CDK4 and 6 inhibitor-containing therapy as a monotherapy. In some other embodiments, the patient may have been administered a prior CDK4 and 6 inhibitor-containing therapy in combination with an additional therapy. In some further embodiments, the prior CDK4 and 6 inhibitor-containing therapy comprises abemaciclib, palbociclib, or ribociclib. In further embodiments, the prior CDK4 and 6 inhibitor-containing therapy comprises abemaciclib. In some further embodiments, the prior CDK4 and 6 inhibitor-containing therapy comprises palbociclib. In yet some further embodiments, the prior CDK4 and 6 inhibitor-containing therapy comprises ribociclib.

In embodiments of this aspect, the patient may have been administered a prior CDK4 and 6 inhibitor-containing therapy in combination with an endocrine therapy. In some embodiments, the prior endocrine therapy comprises tamoxifen. In some embodiments, the prior endocrine therapy comprises an aromatase inhibitor. In some further embodiments, the aromatase inhibitor comprises letrozole, anastrozole, or exemestane.

In further embodiments of the above aspect and embodiments, the patient may have been administered a combination of a CDK4 and 6 inhibitor with an endocrine therapy for the first line (or initial) treatment of HR+, HER2− advanced or metastatic breast cancer. In some embodiments, the first line treatment comprises an endocrine therapy selected from tamoxifen and an aromatase inhibitor. In some embodiments, the first line treatment comprises ribociclib as the CDK4 and 6 inhibitor. In some embodiments, the first line treatment comprises palbociclib as the CDK4 and 6 inhibitor. In some embodiments, the first line treatment comprises abemaciclib as the CDK 4 and 6 inhibitor.

In further embodiments of the above aspect and embodiments, the patient may have been administered a prior CDK4 and 6 inhibitor-containing therapy as a monotherapy. In some other embodiments, the patient may have been administered a prior CDK4 and 6 inhibitor-containing therapy in combination with an additional therapy. In some further embodiments, the prior CDK4 and 6 inhibitor-containing therapy comprises abemaciclib, palbociclib, or ribociclib.

In further embodiments, the prior CDK4 and 6 inhibitor-containing therapy comprises abemaciclib. In some further embodiments, the prior CDK4 and 6 inhibitor-containing therapy comprises palbociclib. In yet some further embodiments, the prior CDK4 and 6 inhibitor-containing therapy comprises ribociclib.

In some further embodiments, the method of treating comprises a patient with advanced or metastatic breast cancer and who has received a prior CDK4 and 6 inhibitor therapy comprising abemaciclib. In such further embodiments, the prior therapy comprising abemaciclib may have been administered: (i) in combination with endocrine therapy (e.g., tamoxifen or an aromatase inhibitor) for the adjuvant treatment in an adult patient with HR+, HER2-node positive, early breast cancer at high risk of recurrence and a Ki-67 score ≥20% as determined by an FDA approved test; (ii) in combination with an aromatase inhibitor as initial endocrine-based therapy for the treatment of postmenopausal women with HR+, HER2− advanced or metastatic breast cancer; (iii) in combination with fulvestrant for the treatment of women with HR+, HER2− advanced or metastatic breast cancer; or (iv) as monotherapy for the treatment of adult patients with HR+, HER2− advanced or metastatic breast cancer with disease progression following endocrine therapy.

In some further embodiments, the method of treating comprises a patient with advanced or metastatic breast cancer and who has received a prior CDK4 and 6 inhibitor therapy comprising palbociclib. In such further embodiments, the prior therapy comprising palbociclib may have been administered: (i) in combination with an aromatase inhibitor as initial endocrine-based therapy for the treatment of postmenopausal women with HR+, HER2− advanced or metastatic breast cancer; or (ii) in combination with fulvestrant for the treatment of women with HR+, HER2− advanced or metastatic breast cancer following endocrine therapy.

In some further embodiments, the method of treating comprises a patient with advanced or metastatic breast cancer and who has received a prior CDK4 and 6 inhibitor therapy comprising ribociclib. In such further embodiments, the prior therapy comprising ribociclib may have been administered: (i) in combination with an aromatase inhibitor as initial endocrine-based therapy for the treatment of pre/perimenopausal or postmenopausal women with HR+, HER2− advanced or metastatic breast cancer; or (ii) in combination with fulvestrant for the treatment of postmenopausal women with HR+, HER2− advanced or metastatic breast cancer, as initial endocrine-based therapy or following disease progression on endocrine therapy.

In some further embodiments, the method according to any of the above aspects and embodiments comprises administering abemaciclib as a 150 mg oral dose twice daily on days 1-28 of each 28 day cycle. In yet further embodiments, the above method comprises administering fulvestrant as a 500 mg intramuscular dose on day 1 and 15 of a first 28 day cycle (cycle 1), and on day 1 of a second and any subsequent 28 day cycle (cycle 2 and subsequent cycles).

In another aspect, the disclosure provides a method of treating a patient with hormone receptor-positive (HR+), human epidermal growth factor receptor 2-negative (HER2−) advanced or metastatic breast cancer previously treated with a CDK4 and 6 inhibitor-containing therapy selected from abemaciclib, ribociclib, and palbociclib, the method comprising administering to the patient an effective amount of abemaciclib in combination with fulvestrant, wherein abemaciclib is administered twice daily as a 150 mg oral dose on days 1-28 of each 28 day cycle, and wherein fulvestrant is administered as a 500 mg intramuscular dose on day 1 and 15 of a first 28 day cycle, and on day 1 of a second and any subsequent 28 day cycle.

In any of the above aspects and embodiments, the method may comprise administering the CDK4 and 6 inhibitor in simultaneous, separate, or sequential combination with fulvestrant.

In any of the above aspects and embodiments, the method may be administered for a time sufficient to provide the patient progression-free survival.

In any of the above aspects and embodiments, the patient is human. In further embodiments, the patient may be an adult male, or an adult premenopausal, perimenopausal, or postmenopausal woman.

In another aspect, the disclosure provides a CDK4 and 6 inhibitor for use in simultaneous, separate or sequential combination with fulvestrant, in the treatment of a patient with hormone receptor positive (HR+), human epidermal growth factor receptor 2 negative (HER2−), advanced or metastatic breast cancer wherein the patient had received a prior CDK4 and 6 inhibitor-containing therapy.

In another aspect, the disclosure provides a combination comprising a therapeutically effective amount of CDK4 and 6 inhibitor and fulvestrant for simultaneous, separate or sequential use in providing treatment to a patient with HR+, HER2− advanced or metastatic breast cancer with disease progression on or after a prior therapy of a CDK4 and 6 inhibitor, for a time period sufficient to provide progression free survival.

In another aspect, the disclosure provides a CDK4 and 6 inhibitor for use in simultaneous, separate or sequential combination with fulvestrant for the treatment of a patient with HR+, HER2− advanced or metastatic breast cancer with disease progression on or after a prior therapy of a CDK4 and 6 inhibitor.

In another aspect, the disclosure provides for the use of a CDK4 and 6 inhibitor in the manufacture of a medicament for the treatment of a patient with HR+, HER2− advanced or metastatic breast cancer who has received a prior therapy of a CDK4 and 6 inhibitor, wherein the medicament is to be administered in simultaneous, separate or sequential combination with fulvestrant. In some embodiments, the patient experienced disease progression on the prior therapy of a CDK4 and 6 inhibitor.

In another aspect, the disclosure provides for the use of a CDK4 and 6 inhibitor in the manufacture of a medicament for the treatment of a patient with HR+, HER2− advanced or metastatic breast cancer with disease recurrence on or after a prior therapy of a combination of a CDK4 and 6 inhibitor with endocrine therapy for the adjuvant treatment of early breast cancer, wherein the medicament is to be administered in simultaneous, separate or sequential combination with fulvestrant.

These aspects as well as other aspects and embodiments will be apparent in light of the description that follows.

### DETAILED DESCRIPTION

Methods, uses, and compositions for the treatment of a patient with hormone receptor-positive (HR+), human epidermal growth factor receptor 2-negative (HER2−) advanced or metastatic breast cancer, and who has been previously treated with a CDK4 and 6 inhibitor-containing therapy, comprising administering to the patient a CDK4 and 6 inhibitor in combination with fulvestrant are disclosed herein. While there has been significant interest and an increased use of therapies that include a CDK4 and 6 inhibitor in the treatment of breast cancer, no clinical evidence exists that serves to guide standard of care therapy for patients with relapsed or progressive metastatic disease that occurs on or after an administered CDK4 and 6 containing therapy. For example, the 2020 ESMO and 2021 NCCN clinical guidelines support use of options that include (i) endocrine therapy alone (e.g., aromatase inhibitors (AI), selective estrogen receptor modulators or degraders (SERMs or SERDs)); (ii) endocrine therapy in combination with PI3K pathway blockade (e.g., with everolimus or alpelisib, if an actionable PIK3 (A mutation is detected); (iii) cytotoxic therapy; and (iv) clinical trial participation. As such, very little data exists that serves to identify and define the therapy that may be best for patients having HR+, HER2− advanced or metastatic breast cancer following an adjuvant therapy or first line therapy comprising a CDK4 and 6 inhibitor, either as a monotherapy or in combination with ET. There is an unmet need to improve outcomes for such patients.

In a general aspect, the disclosure relates to a method of treating a patient with hormone receptor-positive (HR+), human epidermal growth factor receptor 2-negative (HER2−) advanced or metastatic breast cancer who has previously been treated with a CDK4 and 6 inhibitor-containing therapy, comprising administering a CDK4 and 6 inhibitor in combination with fulvestrant.

Methods of treatment and methods of treating a patient in accordance with various aspects and embodiments of the disclosure comprise a patient who has received a previous therapy comprising a CDK4 and 6 inhibitor. “Previously treated” or “previous therapy” with a CDK4 and 6 inhibitor in accordance with the aspects and embodiments disclosed herein encompasses any prior administration of a therapy for the treatment of HR+, HER2− breast cancer (e.g., early stage, advanced, or metastatic breast cancer) that comprises a CDK4 and 6 inhibitor. In some embodiments a previous CDK4 and 6 inhibitor-containing therapy comprises treatment of early stage HR+, HER2− breast cancer. In some embodiments a prior CDK4 and 6 inhibitor treatment of early stage HR+, HER2− breast cancer comprises an adjuvant treatment, comprising administering to the patient a CDK4 and 6 inhibitor optionally in combination with an endocrine therapy. In some further embodiments, the prior adjuvant treatment is administered to a patient with node positive, early breast cancer at a high risk of recurrence. In some further embodiments, the prior adjuvant treatment is administered to a patient with early breast cancer with a Ki-67 score of at least 20% or more as determined by an FDA approved test. In some further embodiments, the prior adjuvant treatment is administered to a patient with node positive, early breast cancer at high risk of recurrence as determined by four or more positive axillary lymph nodes (≥4 pALN), or one to three positive axillary lymph nodes (1-3 pALN) and with a tumor grade 3 and/or tumor size ≥5 cm. In some further embodiments, the prior adjuvant treatment is administered to a patient with node positive, early breast cancer at high risk of recurrence as determined by one to three positive axillary lymph nodes (1-3 pALN), a Ki-67 ≥20%, and no grade 3 tumor and tumor size not ≥5 cm.

In some embodiments a previous CDK4 and 6 inhibitor-containing therapy comprises an initial or first line treatment of advanced or metastatic HR+, HER2− breast cancer. In some embodiments the initial or first line treatment of advanced or metastatic HR+, HER2− breast cancer comprises administering to the patient a CDK4 and 6 inhibitor optionally in combination with an endocrine therapy. In some further embodiments the first line treatment is administered with palbociclib in combination with an endocrine therapy. In some further embodiments, the first line treatment is administered with ribociclib in combination with an endocrine therapy. In yet some further embodiments, the first line treatment is administered with abemaciclib in combination with an endocrine therapy.

In some preferred embodiments, a previous CDK4 and 6 inhibitor-containing therapy may be an incomplete course of therapy. In some preferred embodiments, a previous CDK4 and 6 inhibitor-containing therapy may be an incomplete course of therapy wherein disease progression occurs during treatment of advanced or metastatic breast cancer. In some preferred embodiments, a previous CDK4 and 6 inhibitor-containing therapy may be an incomplete course of therapy wherein disease recurs on adjuvant treatment of early breast cancer. In some preferred embodiments, a previous CDK4 and 6 inhibitor-containing therapy may be a completed course of therapy. In some preferred embodiments, a previous CDK4 and 6 inhibitor-containing therapy may be a completed course of therapy wherein disease recurs after adjuvant treatment of early breast cancer. In some embodiments, a previous CDK4 and 6 inhibitor-containing therapy is halted for a period of time sufficient to provide clearance of the previous therapy from the patient (e.g., 1, 2, 3, 4, 5, 6, 7, 10, 14, 21 days or more), before the administration of the sequential CDK4 and 6 inhibitor in accordance with the disclosure.

As used herein, “a CDK4 and 6 inhibitor-containing therapy” refers to a treatment or therapeutic intervention that comprises administration of a CDK4 and 6 inhibitor to a patient. In some embodiments, a CDK4 and 6 inhibitor-containing therapy can comprise administration of the CDK4 and 6 inhibitor as a monotherapy. In some alternative embodiments, a CDK4 and 6 inhibitor-containing therapy can comprise administration of a CDK4 and 6 inhibitor in combination with one or more other active agents. In some further embodiments, the CDK4 and 6 inhibitor-containing therapy comprises a CDK4 and 6 inhibitor in combination with endocrine therapy.

A “CDK4 and 6 inhibitor” or alternatively a “CDK4/6 inhibitor” refers to molecules that inhibit the activity of D-type cyclins (e.g., cyclin D3) and cyclin-dependent kinases (CDK4 and 6) protein complexes (e.g., cyclinD: CDK4 and 6 complexes), and generally function to block transition from the G1 to S phase of cell cycle through inhibition of kinase activity. In some embodiments of the disclosure, the CDK4 and 6 inhibitor is palbociclib, ribociclib, or abemaciclib.

Palbociclib [6-acetyl-8-cyclopentyl-5-methyl-2-{[5-(piperazin-1-yl)pyridine-2-yl]amino}pyrido[2,3,-d]pyrimidin-7 (8H)-one] is indicated for the treatment of HR+, HER2− advanced or metastatic breast cancer (i) in combination with an aromatase inhibitor as an initial endocrine based therapy in postmenopausal women or in men, or (ii) in combination with fulvestrant in patients with disease progression following endocrine therapy. It has the chemical structure:

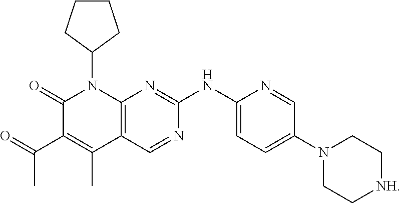 [SMILES: CC(=O)c1c(C)c2cnc(Nc3ccc(N4CCNCC4)cn3)nc2n(C2CCCC2)c1=O]

Palbociclib is taken orally and is available as capsules (125 mg, 100 mg, and 75 mg) with a recommended starting dosage of 125 mg, once daily for 21 days followed by 7 days of off treatment. Palbociclib may be prepared as the free base or as pharmaceutically acceptable salts, including mono- and di-acid addition salts such as, for example, the mono-isethionate salt, polymorphic forms of the isethionate salt, or the hydrochloride salt (see, e.g., WO 2003/062236, WO 2005/005426, WO 2008/032157, U.S. Pat. Nos. 6,936,612; 7,208,489; 7,345,171; 7,456,168; 7,781,583; and 7,863,278). Palbociclib in its free base form may be anhydrous or may contain varying amounts of water or one or more solvents. (see, e.g., U.S. Pat. No. 10,723,730).

Ribociclib [7-cyclopentyl-N,N-dimethyl-2-{[5-(piperazin-1-yl)pyridine-2-yl]amino}-7H-pyrrolo[2,3-d]pyrimidine-6-carboxamide] is indicated for the treatment of HR+, HER2− advanced or metastatic breast cancer (i) in combination with an aromatase inhibitor as an initial endocrine-based therapy in pre/perimenopausal or postmenopausal women, or (ii) in combination with fulvestrant in postmenopausal women as an initial endocrine-based therapy or following disease progression on endocrine therapy. It has the chemical structure:

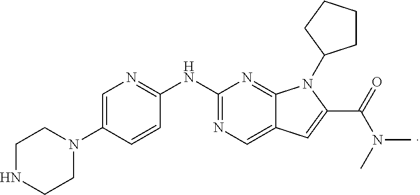 [SMILES: CN(C)C(=O)c1cc2cnc(Nc3ccc(N4CCNCC4)cn3)nc2n1C1CCCC1]

Ribociclib is taken orally and is available as tablets (200 mg, equivalent to 254.40 mg ribociclib succinate) with a recommended starting dosage of 600 mg, (3×200 mg tablets) taken once daily for 21 days followed by 7 days of off treatment. Ribociclib may be prepared as the free base or as pharmaceutically acceptable salts, including as ribociclib succinate (see, e.g., U.S. Pat. Nos. 9,868,739; 9,193,732).

Abemaciclib (LY2835219), [5-(4-ethyl-piperazin-1-ylmethyl)-pyridin-2-yl]-[5-fluoro-4-(7-fluoro-3-isopropyl-2-methyl-3H-benzoimidazol-5-yl)-pyrimidin-2-yl]-amine, its salt forms including the hydrochloride and mesylate salts, and methods of making and using the compound including for the treatment of cancer, in particular, breast cancer are disclosed in WO2010/075074. Methods for using abemaciclib in combination with endocrine therapy for the adjuvant treatment of adult patients diagnosed with HR+, HER2-node positive, early breast cancer at high risk of recurrence and a Ki-67 score ≥20% are disclosed in WO2018/204138. Abemaciclib has the following structure:

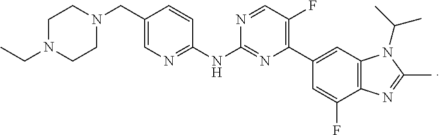 [SMILES: CCN1CCN(Cc2ccc(Nc3ncc(F)c(-c4cc(F)c5nc(C)n(C(C)C)c5c4)n3)nc2)CC1]

Abemaciclib is approved for the treatment of several breast cancer indications, including (i) in combination with endocrine therapy (tamoxifen or an aromatase inhibitor) for the adjuvant treatment of adult patients with HR+, HER2-node-positive, early breast cancer at high risk of recurrence and a Ki-67 score ≥20% as determined by an FDA approved test; (ii) in combination with an aromatase inhibitor as initial endocrine-based therapy for the treatment of postmenopausal women, and men, with HR+, HER2− advanced or metastatic breast cancer; (iii) in combination with fulvestrant for the treatment of adult patients with HR+, HER2− advanced or metastatic breast cancer with disease progression following endocrine therapy; and (iv) as monotherapy for the treatment of adult patients with HR+, HER2− advanced or metastatic breast cancer with disease progression following endocrine therapy and prior chemotherapy in the metastatic setting.

In aspects and embodiments comprising abemaciclib, the following dosing can be employed in accordance with the methods and uses described herein. In some preferred embodiments, abemaciclib, or a pharmaceutically acceptable salt thereof, is administered at a dose of 50 mg to 200 mg twice a day. Also preferably, abemaciclib, or a pharmaceutically acceptable salt thereof, is administered at a dose of 100 mg to 150 mg twice a day. More preferably, abemaciclib, or a pharmaceutically acceptable salt thereof, is administered at a dose of 200 mg twice a day. More preferably, abemaciclib, or a pharmaceutically acceptable salt thereof, is administered at a dose of 150 mg twice a day in a 28-day cycle. More preferably, abemaciclib, or a pharmaceutically acceptable salt thereof, is administered at a dose of 100 mg twice a day in a 28-day cycle. More preferably, abemaciclib, or a pharmaceutically acceptable salt thereof, is administered at a dose of 50 mg twice a day in a 28-day cycle. Preferably, abemaciclib is administered orally. Preferably, abemaciclib is administered by capsule. Also preferably, abemaciclib is administered by tablet.

Fulvestrant is indicated for the treatment of advanced or metastatic breast cancer and is formulated as an injectable (intravenous (IV) or intramuscular (IM)), having the chemical structure:

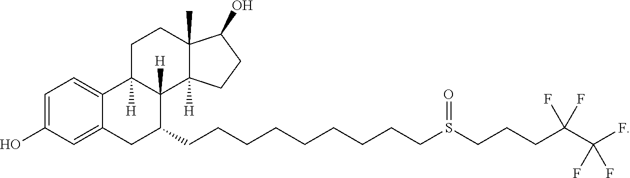

Preferably fulvestrant is administered as described on the approved label, for example, at 500 mg injection once a month, with an additional single 500 mg loading dose on day 15 of the first dose cycle. More preferably fulvestrant is administered as a 500 mg intramuscular dose on day 1 and 15 of a first 28 day cycle, and on day 1 of a second and any subsequent 28 day cycles.

In accordance with the aspects and embodiments described herein, the methods and uses are administered to a patient who has received prior therapy comprising a CDK4 and 6 inhibitor. In some embodiments, the prior therapy comprising a CDK4 and 6 inhibitor is in combination with an endocrine therapy such as, for example, one or more endocrine therapies that may be indicated for the treatment of advanced or metastatic breast cancer with abemaciclib, ribociclib, or palbociclib. As used herein, the term “endocrine therapy” includes tamoxifen or a pharmaceutically acceptable salt thereof, anastrozole, letrozole, or exemestane. In some embodiments, an endocrine therapy can include fulvestrant.

In such embodiments, the endocrine therapy is previously administered in accordance with the guidance and direction on the approved label of the particular endocrine therapy. For example, tamoxifen or a pharmaceutically acceptable salt thereof may be administered at 20-40 mg/day. For doses over 20 mg, the dose should be administered in a divided dose of morning and evening. Doses are preferably oral. For example, anastrazole may be administered at 1 mg/day. Doses are preferably oral. For example, letrozole may be administered at 2.5 mg/day. Doses are preferably oral. For example, exemestane may be administered at 25 mg/day. Doses are preferably oral.

Thus, the previously administered CDK4 and 6 therapy can be administered as described on the approved label for the particular CDK4 and 6 inhibitor and in combination with an endocrine therapy according to the approved label for the particular endocrine therapy.

Generally, and as one of ordinary skill in the art will appreciate, a CDK4 and 6 inhibitor in accordance with the aspects and embodiments of the disclosure may be prepared and administered as the inhibitor compound, or as a pharmaceutically acceptable salt thereof. In some embodiments, abemaciclib may be prepared and/or administered as the free base. In some other embodiments, abemaciclib may be prepared and/or administered as a pharmaceutically acceptable salt such as, for example, the hydrochloride or mesylate salt. In some embodiments, ribociclib may be prepared and/or administered as the free base. In some other embodiments, or ribociclib may be prepared and/or administered as a pharmaceutically acceptable salt such as, for example, ribociclib succinate. In some embodiments, palbociclib may be prepared and/or administered as the free base. In some other embodiments, palbociclib may be prepared and/or administered as a pharmaceutically acceptable salt such as, for example, the isethionate or the hydrochloride salt. In addition to the preparation of certain pharmaceutically acceptable salts of the CDK4 and 6 inhibitors referred to herein, the formation of pharmaceutically acceptable salts is generally well known. See, for example, Gould, P. L., “Salt selection for basic drugs,” International Journal of Pharmaceutics, 33:201-217 (1986); Bastin, R. J., et al. “Salt Selection and Optimization Procedures for Pharmaceutical New Chemical Entities,” Organic Process Research and Development, 4:427-435 (2000); and Berge, S. M., et al., “Pharmaceutical Salts,” Journal of Pharmaceutical Sciences, 66:1-19, (1977).

As used herein, the term “patient” refers to a human. In particular embodiments, the patient may be an adult male, or an adult premenopausal, perimenopausal, or postmenopausal woman, who has or is diagnosed with HR+, HER2− advanced or metastatic breast cancer. In yet further embodiments the patient has HR+, HER2− advanced or metastatic breast cancer and has been previously treated with a CDK4 and 6 inhibitor-containing therapy, in accordance with the disclosure.

As used herein, the terms “cancer” and “cancerous” refer to or describe the physiological condition in patients that is typically characterized by unregulated cell proliferation.

As used herein, the term “effective amount” refers to the amount or dose of a CDK4 and 6 inhibitor (such as e.g., abemaciclib, palbociclib, or ribociclib) and the amount or dose of fulvestrant which provides an effective response in the patient under treatment.

As used herein, the term “effective response” of a patient or a patient's “responsiveness” to treatment with a combination of agents refers to the clinical or therapeutic benefit imparted to a patient upon administration of a CDK4 and 6 inhibitor (such as e.g., abemaciclib, palbociclib, or ribociclib) or a pharmaceutically acceptable salt thereof, and fulvestrant. For example, an effective response can include, but is not limited to, any one or more of progression free survival (PFS) (e.g., based on investigator assessment or blinded independent review (BICR)), overall survival (OS), objective response rate (ORR), clinical benefit rate (CBR), disease control rate (DCR), duration of response (DoR), safety, patient-reported outcomes (PRO), pharmacokinetics (PK), or a best overall response (BOR) that may include complete response (CR), partial response (PR), or stable disease (SD). Accordingly, an effective response is not limited to curing, eliminating, or ameliorating the disease or the clinical symptoms associated with the disease.

As used herein, the term “in combination with” refers to the administration of a CDK4 and 6 inhibitor (e.g., abemaciclib), or a pharmaceutically acceptable salt thereof, and an endocrine therapy (e.g., fulvestrant) either simultaneously or sequentially in any order, such as for example, at repeated intervals as during a standard course of treatment for a single cycle or more than one cycle, such that one agent can be administered prior to, at the same time, or subsequent to the administration of the other agent, or any combination thereof.

As used herein, the term “early stage” means cancers that may have spread to nearby lymph nodes but not to distant parts of the body. In various embodiments of the methods and uses described herein, treatment of early stage breast cancer can be referred to as “adjuvant treatment”.

As used herein, the term “advanced” or “metastatic” means cancers that have spread to one or more parts of the body that were not the site of the original cancerous tissue. In various embodiments of the methods and uses described herein, a first treatment of advanced or metastatic breast cancer with a CDK4 and 6 inhibitor therapy can be referred to as “initial treatment” or “first line treatment”.

As used herein, the term “treating”, or “treatment”, means the administration of a drug or drugs to a patient. The terms can also be used in connection with diminishing, inhibiting, reducing, arresting, or ameliorating the disease, or to delay the onset of the biological manifestation of disease progression.

As used herein, the term “adjuvant treatment” means the administration of a drug or drugs to a patient after surgical resection of one or more cancerous tumors, where all detectable and resectable disease (for example, cancer) has been removed from the patient, but where there remains a statistical risk of relapse due to occult disease, for the purpose of diminishing the likelihood or the severity of reoccurance of the disease, or to delay the onset of the biological manifestation of the reoccurance of the disease.

“Ki67 antigen” or simply “Ki67” (also known as antigen identified by monoclonal antibody Ki-67) means a nuclear protein encoded by the MK167 gene that is expressed in all phases of the cell cycle other than the G₀ phase and has been reported as an independent prognostic factor in early breast cancer (Dowsett et al. 2011). In HR+ breast cancer, patients with high levels (e.g., a threshold value of Ki67 within the range of 20% to 29%) of Ki67 have been shown to have higher disease recurrence rates while receiving adjuvant endocrine therapy following surgery.

The following are further numbered aspects of the invention:

1. The use of a CDK4 and 6 inhibitor in the manufacture of a medicament for the treatment of a patient with hormone receptor positive (HR+), human epidermal growth factor receptor 2 negative (HER2−), advanced or metastatic breast cancer after a prior CDK4 and 6 inhibitor-containing therapy, wherein the medicament is to be administered in simultaneous, separate or sequential combination with fulvestrant.

2. The use according to aspect 1, wherein the previous CDK4 and 6 inhibitor-containing therapy comprised abemaciclib, palbociclib, or ribociclib.

3. The use according to aspect 2, wherein the previous CDK4 and 6 inhibitor-containing therapy comprised abemaciclib.

4. The use according to aspect 2, wherein the previous CDK4 and 6 inhibitor-containing therapy comprised palbociclib.

5. The use according to aspect 2, wherein the previous CDK4 and 6 inhibitor-containing therapy comprised ribociclib.

6. The use according to any of aspects 1-5, wherein the prior CDK4 and 6 inhibitor-containing therapy was a combination of a CDK 4/6 inhibitor with endocrine therapy for the adjuvant treatment of early breast cancer.

7. The use according to aspect 6, wherein the prior adjuvant treatment comprised an endocrine therapy selected from tamoxifen and an aromatase inhibitor.

8. The use according to any one of aspects 6-7, wherein the prior adjuvant treatment was administered to a patient at a high risk of recurrence.

9. The use according to any one of aspects 6-8, wherein the prior adjuvant treatment was administered to a patient with a Ki-67 score ≥20% as determined by an FDA approved test.

10. The use according to any of aspects 1-5, wherein the previous CDK4 and 6 inhibitor-containing therapy was a combination of a CDK 4/6 inhibitor with endocrine therapy for the initial treatment of advanced or metastatic breast cancer.

11. The use according to any one of aspects 1-5, wherein the previous CDK4 and 6 inhibitor-containing therapy was a combination of a CDK 4/6 inhibitor with an aromatase inhibitor for the initial treatment of advanced or metastatic breast cancer.

12. The use according to aspect 11, wherein the aromatase inhibitor is selected from letrozole, anastrozole, or exemestane.

13. The use according to any one of aspects 1-12, wherein the CDK4 and 6 inhibitor administered in combination with fulvestrant is selected from abemaciclib, palbociclib, and ribociclib.

14. The use according to aspect 13, wherein the CDK4 and 6 inhibitor is abemaciclib.

15. The use according to aspect 13, wherein the CDK4 and 6 inhibitor is palbociclib.

16. The use according to aspect 13, wherein the CDK4 and 6 inhibitor is ribociclib.

17. The use according to aspect 14, comprising administering abemaciclib as a 150 mg oral dose twice daily on days 1-28 of each 28 day cycle.

18. The use according to aspect 14 or 17, wherein fulvestrant is administered as a 500 mg intramuscular dose on day 1 and 15 of a first 28 day cycle (cycle 1), and on day 1 of a second and any subsequent 28 day cycle (cycle 2 and subsequent cycles).

19. The use of abemaciclib in the manufacture of a medicament for the treatment of a patient with hormone receptor positive (HR+), human epidermal growth factor receptor 2 negative (HER2−), advanced or metastatic breast cancer after a prior CDK4 and 6 inhibitor-containing therapy, wherein abemaciclib is administered twice daily as a 150 mg oral dose on days 1-28 of each 28 day cycle, wherein fulvestrant is administered as a 500 mg intramuscular dose on day 1 and 15 of a first 28 day cycle, and on day 1 of a second and any subsequent 28 day cycle, and wherein the prior CDK4 and 6 inhibitor-containing therapy is a combination of abemaciclib and tamoxifen or an aromatase inhibitor.

20. The use according to any one of aspects 1-19, wherein the administering is for a time sufficient to provide progression-free survival.

21. The use according to any one of aspects 1-20, wherein the patient is an adult man or an adult premenopausal, perimenopausal, or postmenopausal woman.

The following Examples merely serve to illustrate various aspects and embodiments of the disclosure and should not be considered as limiting the scope of the disclosure.

### Example 1. Sequential Treatment with Abemaciclib with Endocrine Therapy Inhibits Cell Proliferation in Cells with Prior Resistance to CDK4 and 6 Inhibitors

Resistant breast cancer cells are prepared by treating breast cancer cell lines with amounts of a CDK4 and 6 inhibitor (either abemaciclib or palbociclib) in combination with 4-OH-tamoxifen (tamoxifen) for 120-144 h. The cells are sorted and screened for resistant cells, defined as geminin positive (GEM+), a marker of S/G2/M cell cycle accumulation. To confirm the resistant phenotype, cell lines are treated with tamoxifen plus the CDK4 and 6 inhibitor used to drive resistance (either abemaciclib or palbociclib).

### Sequential CDK4 and 6 Inhibitor Treatment.

Resistant cancer cells generated by the above method are treated with abemaciclib in combination with an endocrine therapy (ET) comprising either fulvestrant or tamoxifen; or with palbociclib in combination with either fulvestrant or tamoxifen. Cell proliferation and viability are evaluated by Geminin/Ki67, annexin V, and colony formation assays. Mechanisms of resistance and the effects of sequential treatment of the CDK4 and 6 inhibitor in combination with either fulvestrant or tamoxifen are characterized by western blot and RNAseq analysis.

Cancer cell lines that are resistant to palbociclib and tamoxifen show decreased % GEM+ and colony formation ability, decreased Ki67 levels, and increased apoptosis when sequentially treated with a combination of abemaciclib and ET. In contrast, cell lines resistant to abemaciclib and tamoxifen do not show similar inhibition effects when sequentially treated with a combination of palbociclib and ET. Western blot analysis demonstrates that both palbociclib and abemaciclib-resistant cells exhibit increases in levels of CDK6 and pERK relative to control. Treatment of palbociclib-resistant cells with abemaciclib and ET is effective to decrease FOXMI, a regulator of senescence and apoptosis, as well as to decrease cyclin A, a marker of mitosis, which is consistent with a decreased % GEM+ cell subpopulation in palbociclib and ET-resistant cells. Treatment of abemaciclib-resistant cells with a combination of palbociclib and ET do not exhibit similar effects.

Cancer cell lines that are resistant to palbociclib and tamoxifen show proliferation decrease, decreased pRb signaling, and androgen response induction when sequentially treated with a combination of abemaciclib and ET. Treatment of abemaciclib-resistant cells with a combination of palbociclib and ET do not exhibit similar effects on proliferation, pRb signaling, or androgen response.

This example demonstrates that cancer cells having resistance to a combination CDK4 and 6 inhibitor with ET (e.g., tamoxifen) are susceptible to a sequential treatment with abemaciclib in combination with ET, and provides a therapeutic option for patients who have advanced or metastatic breast cancer that show resistance to an on-going or prior CDK4 and 6 inhibitor-containing therapy.

### Example 2. Randomized, Phase 3 Study of Fulvestrant with or without Abemaciclib in Participants with HR+, HER2− Advanced or Metastatic Breast Cancer with Disease Progression on or after Either an Adjuvant or a First-Line Treatment with a CDK4 and 6 Inhibitor Plus Endocrine Therapy

As detailed below, a clinical trial is conducted as a Phase 3, global, multicenter, randomized, double-blind, placebo-controlled study in participants with HR+, HER2− advanced or metastatic breast cancer. The study will enroll adults who experienced disease progression on a CDK4 and 6 inhibitor and an aromatase inhibitor (AI) therapy in the first-line setting (initial therapy for advanced or metastatic breast cancer), or recurrence on or after a CDK4 and 6 inhibitor with endocrine therapy (ET) in the adjuvant setting.

The trial will include approximately 350 participants that will be randomly assigned to one of two intervention arms that include an investigational arm A: abemaciclib and fulvestrant and a control arm B: placebo and fulvestrant.

TABLE 1
Study Arm Interventions.

| | Arm A | | Arm B | |
|---|---|---|---|---|
| Treatment | Abemaciclib | Fulvestrant | Placebo | Fulvestrant |
| Dose | 150 mg | 500 mg | Matched to | 500 mg |
| | | | abemaciclib | |
| Route | PO | IM | PO | IM |
| Schedule | BID on Days | C1D1 and | BID on Days | C1D1 and |
| | 1-28 of each | C1D15, then | 1-28 of each | C1D15, then |
| | cycle | on Day 1 of | cycle | on Day 1 of |
| | | Cycle 2 and | | Cycle 2 and |
| | | subsequent | | subsequent |
| | | cycles | | cycles |

Abbreviations:
BID = twice daily;
C = cycle;
D = day;
IM = intramuscular administration;
mg = milligrams;
PO = orally administered.

Patient randomization will be 1:1 and stratified by factors including: geography (USA, East Asia, or Other (including EU)); presence of visceral metastases (yes or no); and duration on prior CDK4 and 6 inhibitor-based regimen (2 levels, based on adjuvant/first line therapy), wherein duration of <12 months if prior treatment was in metastatic setting; or disease recurrence during CDK4 and 6 inhibitor-based regimen if treated in adjuvant setting, or duration of ≥12 months if prior treatment was in first line/metastatic setting; or disease recurrence after completing CDK4 and 6 inhibitor-based regimen if treated in adjuvant setting.

The primary endpoint will be progression free survival (PFS) based on investigator assessment. Participants will be treated until disease progression or other discontinuation criteria are met. Secondary endpoints will include overall survival (OS), PFS by blinded independent central review (BICR), objective response rate (ORR), clinical benefit rate (CBR), disease control rate (DCR), duration of response (DoR), Safety, patient-reported outcomes (PRO), and pharmacokinetics (PK).

The primary endpoint, PFS by investigator assessment, is defined as the time from randomization until the first occurrence of documented disease progression as determined by investigator assessment per RECIST 1.1, or death from any cause in the absence of documented progressive disease. PFS will be compared between treatment arms using a stratified log-rank test, stratified by the randomization strata. The corresponding hazard ratio between treatment arms will be estimated using a stratified Cox regression model (Cox 1972), stratified by randomization strata. PFS curves, median PFS, and PFS rates at various time points with 95% CI for each treatment arm will be estimated using the Kaplan-Meier method (Kaplan and Meier 1958).

An interim efficacy analysis of PFS will occur once approximately 176 PFS events in total have been observed with Type I error controlled via the sequential monitoring approach of DeMets and Lan (1994) with the O'Brien-Fleming type spending function.

Secondary Endpoints. Objective Response Rate (ORR), Disease Control Rate (DCR), Clinical Benefit Rate (CBR).

Objective response rate (ORR) is defined as the number of participants who achieve a best overall response (BOR) of complete response (CR) or partial response (PR) divided by the total number of participants randomized to the corresponding treatment arm. Confirmation of CR and PR is not required.

Disease control rate (DCR) is defined as the number of participants who achieve a BOR of CR, PR, or stable disease (SD) divided by the total number of participants randomized to the corresponding treatment arm (ITT population). Confirmation of CR and PR is not required.

Clinical benefit rate (CBR) is defined as the number of participants who achieved a BOR of CR or PR, or SD≥6 months, divided by the total number of participants randomized to the corresponding treatment arm (ITT population). Confirmation of CR and PR is not required. For each of these rates, point estimates and 95% confidence intervals (using the normal approximation to the binomial) will be calculated by treatment arm. Stratified tests comparing these rates between treatment arms will be conducted using the Cochran-Mantel-Haenszel test adjusting for the randomization strata.

Duration of response (DoR) is defined as the time from the date that measurement criteria for CR or PR (whichever is first recorded) are first met until the first date that disease is recurrent or documented disease progression is observed, per RECIST 1.1 criteria, or the date of death from any cause in the absence of documented disease progression or recurrence.

Overall survival (OS) is secondary endpoint of particular focus for this study and is defined as the time from randomization until death from any cause. If the patient is alive or lost to follow-up at the time of data analysis, OS data will be censored on the last date the patient is known to be alive.

TABLE 2
Summary of Objectives and Endpoints.

| Objectives | Endpoints |
|---|---|
| Primary | |
| To compare the efficacy | PFS as determined by |
| of fulvestrant with or | investigator assessment |
| without abemaciclib | using RECIST 1.1 |
| Secondary | |
| To further compare the efficacy | OS |
| of fulvestrant with or | PFS by BICR |
| without abemaciclib | ORR |
| | CBR |
| | DCR |
| | DoR |
| To further characterize the | Safety—including but not |
| safety profile of abemaciclib in | limited to TEAEs, SAEs, |
| combination with fulvestrant | deaths, and clinical laboratory |
| | abnormalities |
| To compare PRO measures | Time to worsening in worst pain |
| of fulvestrant with or | via the mBPI-SF worst pain item |
| without abemaciclib | Time to deterioration in physical |
| | function via the EORTC IL-19 |
| To characterize the | Concentrations of abemaciclib |
| pharmacokinetics (PK) of | |
| abemaciclib in combination | |
| with fulvestrant | |

Abbreviations:
BICR = blinded independent central review;
CBR = clinical benefit rate;
DCR = disease control rate;
DoR = duration of response;
EORTC IL-19 = European Organisation for Research and Treatment of Cancer Item Library 19;
mBPI-SF = modified Brief Pain Inventory-short form;
ORR = objective response rate;
OS = overall survival;
PRO = patient-reported outcome;
PFS = progression-free survival;
RECIST = Response Evaluation Criteria in Solid Tumors;
SAE = serious adverse event;
TEAE = treatment-emergent adverse event.

A treatment cycle will be defined as an interval of 28 days. The 28-day cycle length will be maintained throughout the treatment phase regardless of dose interruptions. Participants will begin dosing assigned treatment on C1D1. Every attempt will be made to maintain a 28-day +/−7-day cycle for fulvestrant administration. When delays are required, doses will be resumed at earliest medically appropriate opportunity based on investigator judgment. Additional clinic visits may be required for administration. Treatment will continue until progression, unacceptable toxicity, or other discontinuation criteria are met.

Abemaciclib. For the approved indication in HR+, HER2-MBC, the recommended starting dose of abemaciclib in combination with fulvestrant is 150 mg BID, which is based on the Phase 3 study, MONARCH 2 (Sledge et al. 2017), in which abemaciclib 150 mg BID in combination with fulvestrant exhibited a manageable safety profile and resulted in clinically meaningful PFS and OS benefit compared to fulvestrant/placebo in patients with HR+, HER2− advanced or metastatic breast cancer (Verzenio package insert, 2019; Verzenios SmPC, 2018).

Fulvestrant. The recommended dose of fulvestrant in combination with abemaciclib is consistent with the approved monotherapy dose of fulvestrant. Study participants will receive fulvestrant 500 mg IM on Days 1 and 15 of Cycle 1, then on Day 1 of Cycle 2 and beyond according to the dosing information provided in the approved local label.

General Dosing. Assignment to either abemaciclib (Arm A) or placebo (Arm B) will be blinded to investigators and participants. Blinded study drug will be administered at a starting dose of 150 mg twice daily, and it is provided as 50 mg tablets. Blinded study drug should be taken twice daily (with at least approximately 6 hours separating doses) at the same time each day with 6-8 ounces of water. Participants should be instructed to swallow tablets whole and not chew or crush them.

During the on-study treatment period, participants will return to clinic every 2 weeks (14±3 days) for the first 2 cycles, and then monthly (28±7 days) starting with Cycle 3 until start of short-term follow-up. The duration of this study period is not pre-defined as patients will remain on treatment until disease progression or discontinuation for any reason.

Tumor Response. Tumor response per RECIST 1.1 should be assessed approximately every 8 weeks for the first 12 months (relative to Cycle 1 Day 1), and thereafter approximately every 12 weeks until the participant has objective disease progression, death, or study completion (following evaluation of final OS data).

Short- and Long-Term Follow-Up. Participants discontinuing study intervention will return for an in-clinic short-term follow-up visit. The short-term follow-up visit will take place 30 days (+7 days) after the decision is made to discontinue all study treatment. After the short-term follow-up visit, all participants will enter the long-term follow-up period. Long-term follow-up begins the day after the short-term follow-up visit is completed and continues until participant's death, withdrawal from study, or study completion. Long-term follow-up visits should occur approximately every 2-3 months (Q60-90D) during long-term follow-up. The duration of this study period is not pre-defined as participants will remain in long-term follow-up until death, withdrawal from study, or study completion.

Treatment in the investigation arm (combination of abemaciclib and fulvestrant) is expected to be well tolerated and to achieve a statistically significant primary endpoint and/or delay disease progression. Prolonged disease control may also delay the need for cytotoxic chemotherapy.

### Participant Inclusion Criteria.

Participants who are eligible to be included in the study will fall within all of the following inclusion criteria:

Age. ≥18 years of age (or of an acceptable age according to local regulations, whichever is older) at the time of signing the informed consent.

Type of Participant and Disease Characteristics. Participants will have a diagnosis of HR+, HER2− breast cancer. To fulfill the requirement of HR+ disease immunohistochemistry (IHC) must show expression of at least one of the hormone receptors (estrogen receptor [ER] or progesterone receptor [PgR]) as defined in the relevant American Society of Clinical Oncology (ASCO)/College of American Pathologists (CAP) Guidelines (Hammond et al. 2010). To fulfill the requirement of HER2-disease: IHC or in-situ hybridization must not demonstrate, at initial diagnosis or upon subsequent biopsy, overexpression of HER2 as defined in the relevant ASCO/CAP Guidelines (Wolff et al. 2018).

A participant will have either advanced disease not amenable to curative surgical treatment or metastatic disease.

A participant will have radiologic evidence of disease progression or recurrence either (a) on treatment with a CDK4 and 6 inhibitor (palbociclib, ribociclib, or abemaciclib) plus AI as initial therapy for advanced disease, or on or after treatment with a CDK4 and 6 inhibitor (palbociclib, ribociclib, or abemaciclib) plus ET administered as adjuvant therapy for early-stage breast cancer.

A participant will have either measurable disease or non-measurable but evaluable disease. Measurable, non-measurable, and evaluable disease are defined according to the Response Evaluation Criteria in Solid Tumors (RECIST Version 1.1 [v1.1], Eisenhauer et al. 2009).

A participant will have a performance status (PS) of 0 or 1 on the Eastern Cooperative Oncology Group (ECOG) scale (Oken et al. 1982).

A participant must be deemed appropriate for treatment with ET.

A participant will have discontinued previous treatments and recovered from the acute effects of therapy to at least Grade 1, except for residual alopecia and peripheral neuropathy, with the therapy washout periods required prior to receiving study drug summarized in Table 3.

TABLE 3
Prior treatment washout periods

| | Length of Time Prior to |
|---|---|
| Previous Treatment | First Dose of Study Drug |
| CDK4 and 6 inhibitor | ≥7 days |
| Endocrine therapy | ≥7 days or 5 half-lives, |
| | whichever is shorter |
| Radiotherapy | ≥14 days |
| Major surgery | ≥14 days |

Participants must have adequate organ function, as summarized in Table 4:

TABLE 4
Hematologic and hepatic function thresholds

| System | Laboratory Value |
|---|---|
| Hematologic | |
| ANC | ≥1.5 × 10⁹/L |
| Platelets | ≥100 × 10⁹/L |
| Hemoglobin | ≥8 g/dL |
| Hepatic | |
| Total bilirubin | ≤1.5 × ULN |
| | Participants with Gilbert's syndrome with a total |
| | bilirubin ≤2.0 × ULN and direct bilirubin within |
| | normal limits are permitted |
| ALT and AST | ≤3 × ULN |

Note:
transfusions to increase a patient's hemoglobin or initiation of erythropoietin or G-CSF therapy to meet enrollment criteria are not allowed in the 14 days preceding the first dose of study drug.
Abbreviations:
ALT = alanine aminotransferase;
ANC = absolute neutrophil count;
AST = aspartate aminotransferase;
G-CSF = granulocyte colony-stimulating factor;
ULN = upper limit of normal.

Male participants must agree to use hormone suppression (received monthly and initiated at least 28 days prior to Cycle 1 Day 1) with a gonadotropin-releasing hormone agonist such as goserelin or leuprolide. Males are eligible to participate if they agree to refrain from donating sperm during the treatment period. Contraception requirements for male participants receiving fulvestrant should follow the approved local label.

Female participants must have postmenopausal status due to either surgical/natural menopause or ovarian suppression (received monthly and initiated at least 28 days prior to Cycle 1 Day 1) with a gonadotropin-releasing hormone agonist, such as goserelin or leuprolide. Postmenopausal status due to surgical/natural menopause requires at least 1 of the following: (a) prior bilateral oophorectomy, (b) age ≥55 years and amenorrhoeic for at least 12 months or with a diagnosis of menopause, or (c) age ≥40 and <55 years, amenorrhoeic for at least 12 months (in the absence of chemotherapy, tamoxifen, toremifene, or ovarian suppression), and FSH in the postmenopausal range (≥40 mIU/mL).

Women of childbearing potential (WOCBP) must test negative for pregnancy prior to initiation of treatment with a negative serum pregnancy test at the screening visit, followed by a negative urine pregnancy test within 48 hours prior to first exposure to study drug. WOCBP must agree to use 2 forms of effective contraception where at least one form must be highly effective (less than 1% failure rate) to prevent pregnancy while receiving study treatment, for 3 weeks after the last dose of blinded study drug and for 2 years after the last dose of fulvestrant (or according to local approved fulvestrant label).

### Exclusion Criteria

Participants are excluded if they have certain medical conditions or are receiving certain prior or concomitant therapy. Participants are excluded if they are currently enrolled in any other clinical study involving an investigational product or any other type of medical research judged not to be scientifically or medically compatible with this study. Women who are pregnant or breastfeeding are excluded. Any patient who has a known or suspected hypersensitivity reactions or intolerance to the study drug or to any of the excipients (e.g., lactose), unless otherwise deemed appropriate by the investigator.

### Exclusionary Medical Conditions Include Participants Who:

Have visceral crisis, lymphangitic spread, or leptomeningeal carcinomatosis. Visceral crisis is not the mere presence of visceral metastases but implies severe organ dysfunction as assessed by symptoms and signs, laboratory studies, and rapid progression of the disease.
Have symptomatic or untreated central nervous system (CNS) metastasis. Participants with treated CNS metastases are eligible if
   a. they completed prior therapy (including radiation and/or surgery) ≥28 days prior to first dose of study treatment, and
   b. they have not received corticosteroids and/or anticonvulsants for at least 14 days prior to first dose of study treatment, and
   c. their disease is both asymptomatic and radiographically stable by repeat imaging for at least 28 days prior to consent (repeat imaging should be performed during study screening).
Have a history within the last 12 months of any of the following conditions: syncope of cardiovascular etiology, ventricular arrhythmia of pathological origin (including, but not limited to, ventricular tachycardia and ventricular fibrillation), or sudden cardiac arrest. Exception: patients with controlled atrial fibrillation for >30 days prior to randomization are eligible.
Have serious preexisting medical condition(s) that, in the judgment of the investigator, would preclude participation in this study (such as severe renal impairment [for example, estimated creatinine clearance <30 mL/min], active symptoms of ILD/pneumonitis, severe dyspnea at rest or requiring oxygen therapy, history of major surgical resection involving the stomach or small bowel, or preexisting Crohn's disease or ulcerative colitis or a preexisting chronic condition resulting in clinically significant diarrhea).
Have a history of any other cancer (except nonmelanoma skin cancer or carcinoma in-situ of the cervix), unless in complete remission with no therapy for a minimum of 3 years
Have a known active systemic infection (for example, bacterial, fungal, or detectable viral infection requiring systemic therapy).
   a) Participants with uncontrolled human immunodeficiency virus (HIV) infection or an acquired immunodeficiency syndrome (AIDS) defining illness are not eligible. Participants with known HIV infection and CD4+ T-cell (CD4+) counts ≥350 cells/μL are eligible.
   b) Participants with hepatitis B are not eligible unless viral load is below the level of quantification.
   c) Participants with known hepatitis C are not eligible unless they have completed curative anti-viral therapy and viral load is below the level of quantification.
   d) Screening for HIV, coronavirus disease 2019 (COVID-19), hepatitis B, or hepatitis C is not required.

### Exclusionary Prior or Concomitant Therapy Include Participants Who:

Have received any intervening line of systemic therapy between disease recurrence/progression and study screening.
Have received more than 1 line of therapy for advanced or metastatic disease.
Have received prior treatment with chemotherapy for MBC.
Have received prior treatment with any CDK4 and 6 inhibitor-based regimen other than those specified. Prior treatment with a CDK4 and 6 inhibitor in more than 1 setting (e.g., adjuvant and then metastatic) is not permitted.
Have received prior treatment with fulvestrant, any investigational ER-directed therapy (including SERDs and non-SERDs), any PI3K-, mTOR-, or AKT-inhibitor.
Have known pathogenic germline mutations appropriate for a PARP inhibitor, in regions where these therapies are approved and available, per investigator's discretion.
Have initiated bisphosphonates or approved RANK ligand (RANK-L) targeted agents (e.g. denosumab)<7 days prior to randomization.
Are receiving concurrent exogenous reproductive hormone therapy (for example, birth control pills, hormone replacement therapy, or megestrol acetate). Appropriate washout period between last dose and randomization is up to the investigator's medical judgment (for example, applying 7 days or 5 times the half-life elimination rule). Note: topical vaginal estrogen therapy is permitted if all other non-hormonal options are exhausted.
Have received an autologous or allogeneic stem cell transplant.

1. A method of treating a patient with hormone receptor-positive (HR+), human epidermal growth factor receptor 2-negative (HER2−) advanced or metastatic breast cancer previously treated with a CDK4 and 6 inhibitor-containing therapy, the method comprising administering a CDK4 and 6 inhibitor in combination with fulvestrant.

2. The method according to claim 1, wherein the previous CDK4 and 6 inhibitor-containing therapy comprised abemaciclib, palbociclib, or ribociclib.

3. The method according to claim 2, wherein the previous CDK4 and 6 inhibitor-containing therapy comprised abemaciclib.

4. The method according to claim 2, wherein the previous CDK4 and 6 inhibitor-containing therapy comprised palbociclib.

5. The method according to claim 2, wherein the previous CDK4 and 6 inhibitor-containing therapy comprised ribociclib.

6. The method according to claim 1, wherein the prior CDK4 and 6 inhibitor-containing therapy was a combination of a CDK4 and 6 inhibitor with endocrine therapy for the adjuvant treatment of early breast cancer.

7. The method according to claim 6, wherein the prior adjuvant treatment comprised an endocrine therapy selected from tamoxifen and an aromatase inhibitor.

8. The method according to claim 6, wherein the prior adjuvant treatment was administered to a patient at a high risk of recurrence.

9. The method according to claim 6, wherein the prior adjuvant treatment was administered to a patient with a Ki-67 score ≥20% as determined by an FDA approved test.

10. The method according to claim 1, wherein the prior CDK4 and 6 inhibitor-containing therapy was a combination of a CDK4 and 6 inhibitor with endocrine therapy for the initial treatment of advanced or metastatic breast cancer.

11. The method according to claim 10, wherein the previous CDK4 and 6 inhibitor-containing therapy was administered in combination with an aromatase inhibitor.

12. The method according to claim 11, wherein the aromatase inhibitor previously administered in combination with the CDK4 and 6 inhibitor-containing therapy comprised letrozole, anastrozole, or exemestane.

13. The method according to claim 1, wherein the method comprises administering a CDK4 and 6 inhibitor selected from abemaciclib, palbociclib, and ribociclib in combination with fulvestrant.

14. The method according to claim 13, wherein the CDK4 and 6 inhibitor is abemaciclib.

15. The method according to claim 13, wherein the CDK4 and 6 inhibitor is palbociclib.

16. The method according to claim 13, wherein the CDK4 and 6 inhibitor is ribociclib.

17. The method according to claim 14, comprising administering abemaciclib as a 150 mg oral dose twice daily on days 1-28 of each 28 day cycle.

18. The method according to claim 17, wherein fulvestrant is administered as a 500 mg intramuscular dose on day 1 and 15 of a first 28 day cycle (cycle 1), and on day 1 of a second and any subsequent 28 day cycle (cycle 2 and subsequent cycles).

19. A method of treating a patient with hormone receptor-positive (HR+), human epidermal growth factor receptor 2-negative (HER2−) advanced or metastatic breast cancer previously treated with a CDK4 and 6 inhibitor-containing therapy selected from abemaciclib, ribociclib, and palbociclib, the method comprising administering to the patient an effective amount of abemaciclib in combination with fulvestrant, wherein abemaciclib is administered twice daily as a 150 mg oral dose on days 1-28 of each 28 day cycle, and wherein fulvestrant is administered as a 500 mg intramuscular dose on day 1 and 15 of a first 28 day cycle, and on day 1 of a second and any subsequent 28 day cycle.

20. The method according to claim 1, wherein the administering is for a time sufficient to provide progression-free survival.

21. The method according to claim 1, wherein the patient is an adult man or an adult premenopausal, perimenopausal, or postmenopausal woman.

22.-43. (canceled)
